(19) United States
(12) Patent Application Publication
(10) Pub. No.: US 2025/0375382 A1
(43) Pub. Date: Dec. 11, 2025

(54) ORAL PEPTIDE DRUG STRUCTURE AND MANUFACTURING METHOD THEREOF

(71) Applicant: Merdury Biopharmaceutical Corporation, New Taipei (TW)

(72) Inventors: HSIEN-TE LIN, New Taipei (TW); CHAO-YI CHEN, New Taipei (TW)

(21) Appl. No.: 18/736,095

(22) Filed: Jun. 6, 2024

(51) Int. Cl. A61K 9/28; A61K 9/20

(52) U.S. Cl. CPC A61K 9/2886; A61K 9/2009; A61K 9/2013; A61K 9/2054; A61K 9/2893

(57) ABSTRACT

The invention is an oral peptide drug structure mainly comprising: a tablet body and a coating layer covering an outer layer of the tablet body, the tablet body is made of a first excipient mixing with a first penetration accelerator, and the coating layer is made of a second penetration accelerator mixing with a peptide. Through the tablet body design of coating the excipient with the peptide, the oral peptide can be stably absorbed.

### BACKGROUND OF THE INVENTION

### Field of Invention

The invention relates to a peptide-containing tablet body, more particularly to an oral peptide drug structure and a manufacturing method thereof.

### Related Art

Existing oral peptide drug products are scarce due to various difficulties that need to be overcome, so most of them are in the form of injection. The technical problems that need to be overcome are: 1. The peptide has a large molecular weight and is not easily absorbed by the human body through gastrointestinal cells. 2. When the peptide drug is in the stomach, it is easily damaged by pepsin, which destroys the structure and loses its efficacy, resulting in poor bioavailability of the peptide. Peptide drugs currently available in tablet form commercially can overcome the above two problems by evenly mixing the peptide with a suitable excipient. The peptide will be evenly distributed in the tablet. However, the time it can be effectively absorbed in the gastrointestinal tract is short, and it cannot continue to produce medicinal effects in the body.

Therefore, according to current pharmaceutical technology, peptide and excipient can only be mixed evenly and made into a tablet for use. Although it can be absorbed by the human body, its mode of action of medicinal effects is not completely suitable for patients who need to take medicine every day for a long period of time. For example, in patients with diabetes, the plasma drug concentration-time curve fluctuates greatly, which can easily produce side effects in the patient due to the above reason and is unable to achieve stable absorption.

The detailed features and advantages of the invention will be described in detail in the following embodiments. The content is sufficient to enable any person having ordinary skill in the art to understand the technical content of the invention and implement it accordingly, and in accordance with the content, the claims and the drawings disclosed in this specification, any person having ordinary skill in the art can easily understand the related objects and advantages of the invention.

### SUMMARY OF THE INVENTION

A main object of the invention lies in: by forming a tablet body through mixing a first excipient with a first penetration accelerator, and coating an outer layer of the tablet body with a coating layer formed by mixing a second penetration accelerator with a peptide, thereby enabling the tablet body containing the peptide to quickly settle to a bottom of a stomach, and at the same time, to quickly attach to the bottom of the stomach flatly, the peptide is attached to a stomach wall, and the excipient is covered on the peptide. Finally, a pH value of local areas in the stomach can be changed and a permeability of cells can be increased, thereby enabling the peptide to be stably absorbed through the stomach.

In order to achieve the above object, the invention is an oral peptide drug structure comprising: a tablet body made of a first excipient mixing with a first penetration accelerator; and a coating layer made of a second penetration accelerator mixing with a peptide, and the coating layer is coated on an outer layer of the tablet body.

According to one embodiment of the invention, wherein the tablet body is further mixed with a second excipient and a third excipient.

According to one embodiment of the invention, wherein a weight percentage of each of the excipients in the tablet body is preferably: a weight percentage of the first excipient is 2-5% (colloidal silica, SiO2), a weight percentage of the second excipient is 15.54-19.12% (microcrystalline cellulose, MCC) and a weight percentage of the third excipient is 7-12% (croscamellose sodium).

According to one embodiment of the invention, wherein a weight percentage of each of the excipients in the tablet body is more preferably: a weight percentage of the first excipient is 9-12% (colloidal silica, SiO2), a weight percentage of the second excipient is 15.54-19.12% (microcrystalline cellulose, MCC) and a weight percentage of the third excipient is 0.3-1.3% (croscamellose sodium).

According to one embodiment of the invention, wherein an optimal weight percentage of each of the excipients in the tablet body is: a weight percentage of the first excipient is 1˜2% (colloidal silica, SiO2), a weight percentage of the second excipient is 16-20% (microcrystalline cellulose, MCC) and a weight percentage of the third excipient is 8-10% (croscamellose sodium).

According to one embodiment of the invention, wherein a weight percentage of the first penetration accelerator is 62-73%.

According to one embodiment of the invention, wherein the first excipient, the second excipient, the third excipient and the first penetration accelerator are formed viscously and shaped by mixing with a first adhesive, wherein a weight percentage of the first adhesive in the tablet body is 0.13-0.5%.

According to one embodiment of the invention, wherein the peptide in the coating layer is mixed with a fourth excipient, and then mixed with the second penetration accelerator, and then mixed with a fifth excipient already mixed with a sixth excipient.

According to one embodiment of the invention, wherein a weight percentage of each of the excipients in the coating layer is preferably: a weight percentage of the fourth excipient is 22-25% (microcrystalline cellulose, MCC), a weight percentage of the fifth excipient is 2-5% (colloidal silica, SiO2), a weight percentage of the sixth excipient is 5-8% (croscarmellose sodium), and a weight percentage of the peptide in the coating layer is 1.2-13.3%.

According to one embodiment of the invention, wherein a weight percentage of each of the excipients in the coating layer is more preferably: a weight percentage of the fourth excipient is 21-24% (microcrystalline cellulose, MCC), a weight percentage of the fifth excipient is 0.5-3.2% (colloidal silica, SiO2), a weight percentage of the sixth excipient is 7-10% (croscamellose sodium), and a weight percentage of the peptide in the coating layer is 1.2-13.3%.

According to one embodiment of the invention, wherein an optimal weight percentage of each of the excipients in the coating layer is: a weight percentage of the fourth excipient is 22.68% (microcrystalline cellulose, MCC), a weight percentage of the fifth excipient is 2.0% (colloidal silica, SiO2), a weight percentage of the sixth excipient is 8% (croscamellose sodium), and a weight percentage of the peptide in the coating layer is 1.2-13.3%.

According to one embodiment of the invention, wherein the fourth excipient, the fifth excipient, and the sixth excipient are mixed with a second adhesive to form a viscous state and coated on the outer layer of the tablet body, wherein a weight percentage of the second adhesive in the coating layer is 0.13-2.0%.

According to one embodiment of the invention, wherein a manufacturing method of the oral peptide drug structure comprises:
a. mixing the first penetration accelerator with the first excipient to form a first mixture;
b. mixing the second excipient with the third excipient to form a second mixture;
c. mixing the first mixture with the second mixture to form a third mixture;
d. mixing the third mixture with the first adhesive to form a viscous tablet body, and drying it to take shape;
e. mixing the peptide with the second penetration accelerator to form a fourth mixture;
f. mixing the fifth excipient with the sixth excipient to form a fifth mixture;
g. mixing the fourth mixture with the fifth mixture in a dispersion manner to form a sixth mixture; and
h. mixing the sixth mixture with the second adhesive to form a seventh mixture to form a viscous state and coating the tablet body for performing drying operation.

### BRIEF DESCRIPTION OF THE DRAWINGS

FIG. 1 is a schematic structural diagram of an oral peptide of the invention.

FIG. 2 is a flow chart of a manufacturing method of the oral peptide of the invention.

FIG. 3 is a schematic diagram of a state in which the oral peptide containing a peptide of the invention sinks into a stomach.

FIG. 4 is a schematic diagram of a melting state of a coating layer of the invention.

FIG. 5 is a schematic diagram of a state in which the coating layer of the invention is attached to an inner wall of the stomach and a tablet body is disintegrated.

FIG. 6 is a schematic diagram of a state in which the coating layer of the invention is absorbed by the inner wall of the stomach.

FIG. 7 is a schematic diagram of plasma drug concentration curves of a commercially available control drug (RLD) and an experimental tablet body (T2) in experimental animals (dogs).

### DETAILED DESCRIPTION OF THE INVENTION

The following specific embodiments illustrate the implementation mode of the invention. Any person having ordinary skill in the art can easily understand the other advantages and efficacies of the invention from the content disclosed in this specification.

The structures, proportions and sizes depicted in the accompanied drawings in this specification are only used to match with the content disclosed in the specification for understanding and reading by those skilled in the art, and are not intended to limit the conditions that can be implemented by the invention, and therefore are not technically significant. Any structural modifications, changes in proportional relationship or adjustment of sizes without affecting the efficacies generated and the objects achieved by the invention, should still fall within the scope covered by the technical content disclosed by the invention. At the same time, the terms such as “one”, “two”, “above”, etc. cited in this specification are only for clarity of description, and are not used to limit the scope of the invention, changes or adjustments in their relative relationships, without substantial changes to the technical content, should also be regarded as the scope of the invention that can be implemented.

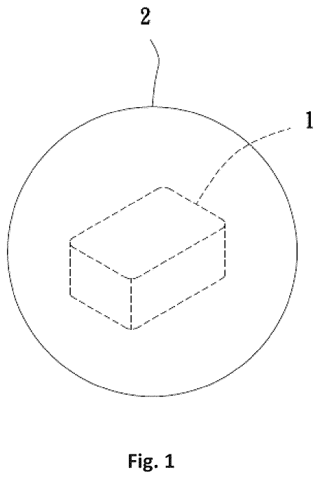

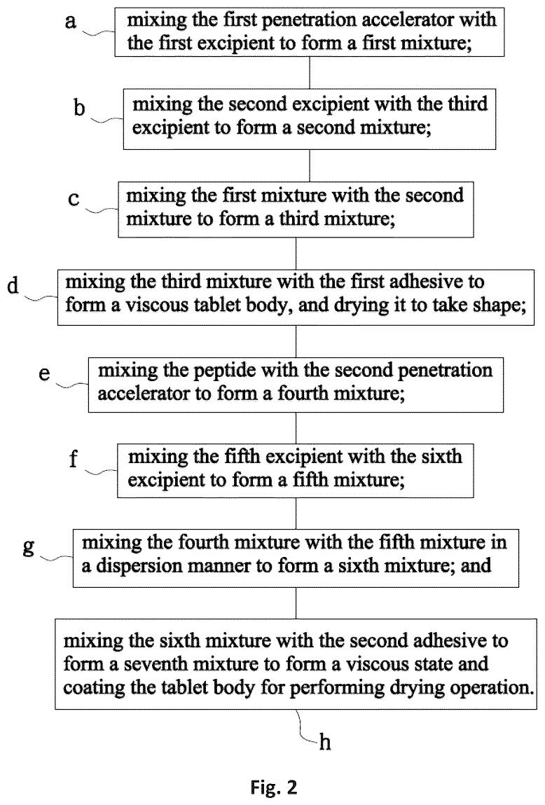

Please refer to FIGS. 1 and 2 respectively for a schematic structural diagram of an oral peptide, and a flow chart of a manufacturing method of the oral peptide of the invention. The invention is an oral peptide drug structure mainly comprising: a tablet body 1 and a coating layer 2 covering an outer layer of the tablet body 1, wherein the tablet body 1 is mainly composed of a first excipient mixing with a first penetration accelerator, wherein the first excipient is a direct tablet binder, in addition to being colloidal silica (SiO2), it can also be replaced by silicon dioxide, silicified microcrystalline cellulose and diclacium phosphate anhydrous. Wherein a main function of the first penetration accelerator is to improve a permeability of cells of the peptide drug, which can be 8-(2-hydroxybenzamide) sodium octanoate (salcaprozate sodium, SNAC), surfactant, bile salt, chelating agent and medium chain fatty acid (MCFA), the surfactant can be medium chain fatty acid, acyl-amino acid, nonionic surfactant, bile salt and acyl-carnitine, the bile salt can be taurodeoxycholate, taurocholate, cholate and deoxycholate, and the chelating agent can be EDTA, etc., and the medium chain fatty acid (MCFA) can be sodium caprate, propylene glycol caprylate, etc., when the first penetration accelerator is 8-(2-hydroxybenzamide) sodium octanoate (salcaprozate sodium, SNAC), it also has an antacid efficacy. Furthermore, the alternative components described above are only for clarity of description and convenience of description and are not limited thereto.

The coating layer 2 is composed of a second penetration accelerator mixing with a peptide 20, and can also contain a fourth excipient according to usage requirements. Wherein the peptide 20 can be a peptide drug, such as semaglutide, etc. The second penetration accelerator mentioned above also functions to improve a permeability of cells of the peptide drug. The selected components of the second penetration accelerator are the same as the first penetration accelerator. If 8-(2-hydroxybenzamide) sodium octanoate (salcaprozate sodium, SNAC) is selected for the second penetration accelerator, it also has an antacid efficacy, it can be used to inhibit gastric enzymes and improve a permeability of cells to achieve an effect of stable absorption.

The tablet body 1 is further added with a second excipient and a third excipient. The second excipient is mainly microcrystalline cellulose, and can also be alternately substituted by corn starch, lactose or mannitol. The third excipient is mainly croscamellose sodium, and can also be alternately substituted by calcium carboxymethyl and sodium starch glycolate. Furthermore, in addition to colloidal silica (SiO2), the first excipient can also be replaced by silicon dioxide, silicified microcrystalline cellulose and diclacium phosphate anhydrous.

A weight percentage of each of the excipients in the tablet body 1 is: a weight percentage of the first penetration accelerator is 62-73%, a weight percentage of the first excipient is 0.5-5%, a weight percentage of the second excipient is 15-23%, and a weight percentage of the third excipient is 7-12%.

If the tablet body 1 has a lowest weight percentage, the insufficient weight percentage will be made up by the second excipient, so a weight percentage of the second excipient is at least more than 15%.

In addition, an overall structure is divided into two layers: the inner tablet body 1 and the outer coating layer 2, each of which is 150˜250 mg.

After mixing of the first excipient, the second excipient, the third excipient and the first penetration accelerator is completed, a third mixture is formed. The third mixture can be formed into a viscous state and shaped by mixing with the first adhesive. Wherein the first adhesive is first prepared with water to form a 0.2% (w/v) first adhesive aqueous solution. The first adhesive can be polyvinylpyrrolidone K90 (PVP K90), and a weight percentage of the first adhesive in the tablet body 1 is 0.13-0.5%. Wherein the first adhesive aqueous solution and the third mixture are stirred and mixed in a ratio of 2:1 until they are viscous, then shaped into a round cake shape and allowed to stand until they solidify and dry, then grind the tablet body 1 to 210.3 mg, an error does not exceed 3%.

In addition to the second penetration accelerator, the peptide in the coating layer 2 can also be mixed with a fourth excipient first, and then mixed with the second penetration accelerator, and then mixed with a fifth excipient already mixed with a sixth excipient. Wherein the fourth excipient is mainly microcrystalline cellulose (MCC), which can be alternately replaced by corn starch, lactose or mannitol, while the fifth excipient is mainly colloidal silica (SiO2), which can be alternately replaced by silicon dioxide, silicified microcrystalline cellulose and diclacium phosphate anhydrous. As for the sixth excipient, it is mainly croscamellose sodium, which can be alternately replaced by calcium carboxymethyl and sodium starch glycolate. Furthermore, the alternative components described above are only for clarity of description and convenience of description and are not limited thereto.

A weight percentage of each of the excipients in the coating layer 2 is: a weight percentage of the fourth excipient is 20-25%, a weight percentage of the fifth excipient is 0.5-5%, a weight percentage of the sixth excipient is 5-9.5%, and a weight percentage of the peptide in the coating layer 2 is 1.2-13.3%.

If the coating layer 2 has a lowest weight percentage, the insufficient weight percentage will be made up by the fourth excipient, so a weight percentage of the fourth excipient is at least more than 20%.

After completing mixing of the fourth excipient, the fifth excipient and the sixth excipient, mix it with the second adhesive to form a viscous shape and coat it on the outer layer of the tablet body 1, wherein the second adhesive can be polyvinylpyrrolidone K90 (PVP K90), and a weight percentage of the second adhesive in the tablet body 1 is 0.13-0.2%. Wherein the second adhesive is first prepared with water to form a 0.2% (w/v) second adhesive aqueous solution, mix it with the powder mixed with the coating layer 2, stir until it becomes viscous, and then coat it on the outer layer of the tablet body 1. After drying, the tablet body 1 is greater than 443.97 mg/tab, and a weight error of each of the tablet bodies 1 does not exceed 3%.

A weight ratio of the tablet body 1 to the coating layer 2 is 50%+5%: 50%+5%, such as: 45%: 55%, 55%: 45%, 50%: 50%, etc.

Steps for making the oral peptide drug structure are as follows:
a. mixing the first penetration accelerator with the first excipient to form a first mixture;
b. mixing the second excipient with the third excipient to form a second mixture;
c. mixing the first mixture with the second mixture to form a third mixture;
d. mixing the third mixture with the first adhesive to form a viscous tablet body;
e. mixing the peptide with the second penetration accelerator to form a fourth mixture;
f. mixing the fifth excipient with the sixth excipient to form a fifth mixture;
g. mixing the fourth mixture with the fifth mixture in a dispersion manner to form a sixth mixture; and
h. mixing the sixth mixture with the second adhesive to form a seventh mixture to form a viscous state and coating the tablet body for performing drying operation. The peptide in the step e can be mixed with the fourth excipient first.

In the step f, the fifth excipient (colloidal silica, SiO2) and the sixth excipient (croscamellose sodium) are mixed to form the fifth mixture in a ratio of: the fifth excipient:the sixth excipient=1:4. As for a dispersion magnification when mixing the fourth mixture with the fifth mixture, since a volume of the fifth mixture when being mixed is much smaller than that of the fourth mixture, a mixing method is to mix the fifth mixture with the fourth mixture at a ratio of 1:2, and continue to mix evenly in this way.

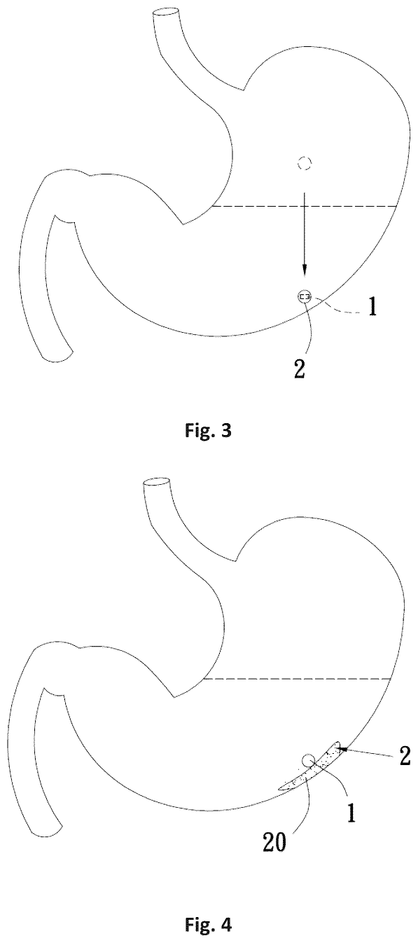

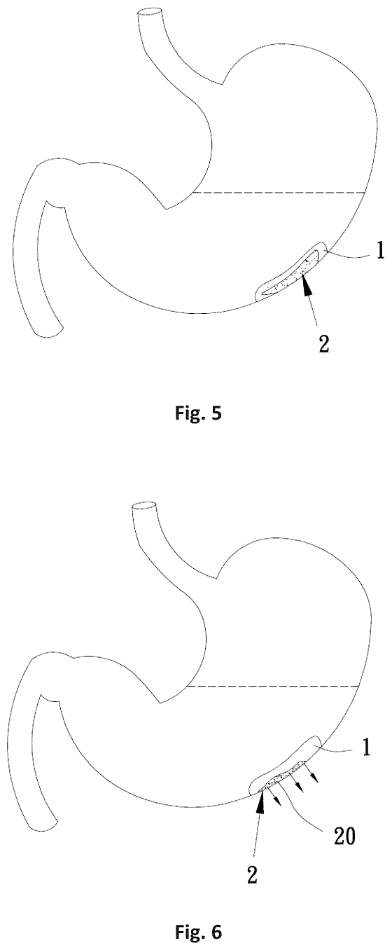

Please refer to FIG. 3, FIG. 4, FIG. 5 and FIG. 6 respectively for a schematic diagram of a state in which the oral peptide containing a peptide of the invention sinks into a stomach; a schematic diagram of a melting state of a coating layer of the invention; a schematic diagram of a state in which the coating layer of the invention is attached to an inner wall of the stomach and a tablet body is disintegrated; and a schematic diagram of a state in which the coating layer of the invention is absorbed by the inner wall of the stomach. It can be known from FIG. 3 that when the whole tablet body 1 enters a stomach, since the tablet body 1 and the coating layer 2 are mixed with adhesive, an overall density is increased, so it can quickly reach a bottom of the stomach without spreading.

Then, it can be known from FIG. 4 that the coating layer 2 located on the outer layer of the tablet body 1 begins to gradually disintegrate and adhere to the bottom of the stomach. At this time, the peptide 20 also adheres to the bottom of the stomach. It can be known from FIG. 5 that, at this time, the tablet body 1 located in the coating layer 2 will float out and begin to disintegrate and spread above the coating layer 2. At this time, when the first penetration accelerator in the tablet body 1 is 8-(2-hydroxybenzamide) sodium octanoate (salcaprozate sodium, SNAC), gastric acid in diffusion areas is acid-base neutralized, which greatly reduces a probability of the peptide 20 being destroyed. At the same time, please refer to FIG. 6, it can be known that the peptide 20 in the coating layer 2 and 8-(2-hydroxybenzamide) sodium octanoate (salcaprozate sodium, SNAC) contact a surface of the bottom of the stomach, which increases a permeability of gastric parietal cells of the peptide 20 through 8-(2-hydroxybenzamide) sodium octanoate (salcaprozate sodium, SNAC).

In order for the peptide 20 to be stably absorbed by an inner wall of the stomach, three problems must be overcome: 1. The peptide 20 is easily destroyed by gastric enzymes. 2. How to make the peptide 20 have good permeability of cells. 3. The peptide 20 cannot easily penetrate cells and be absorbed by the human body. Therefore, the pharmaceutical tablet body 1 of the peptide 20 mixed with 8-(2-hydroxybenzamide) sodium octanoate (salcaprozate sodium, SNAC) can be used to inhibit gastric enzymes and improve a permeability of cells to achieve a stable absorption effect.

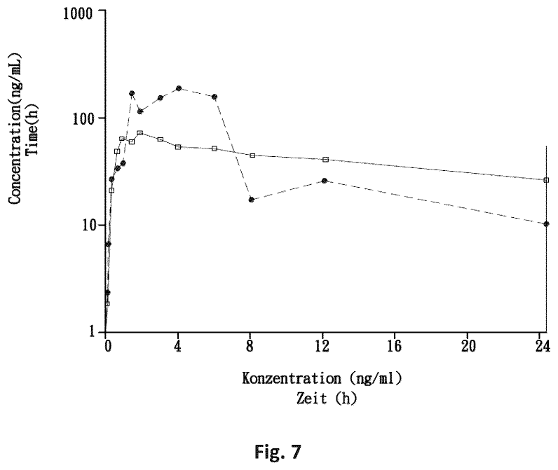

Please refer to FIG. 7 as well for a schematic diagram of plasma drug concentration curves of a commercially available control drug (RLD)—solid dots and an experimental tablet body of the invention (T2)—hollow squares in experimental animals (dogs). For the oral peptide manufactured by the manufacturing method of the oral peptide drug structure of the invention, animal experiments are performed and blood data is obtained, and test items are carried out:

TABLE 1
Table 1: Test data of plasma drug concentration during
sedimentation of the tablet body 1 of the invention
T2

| Time (h) | 7MW1103 | 7MW1131 | 7FW1133 | Mean |
|---|---|---|---|---|
| 0 | 3.415 | 2.135 | blq | 1.850 |
| 0.25 | 9.522 | 9.808 | blq | 6.443 |
| 0.5 | 23.103 | 43.274 | blq | 22.126 |
| 0.75 | 65.906 | 79.835 | blq | 48.580 |
| 1 | 98.162 | 94.740 | blq | 64.301 |
| 1.5 | 107.011 | 73.825 | blq | 60.279 |
| 2 | 135.252 | 87.114 | blq | 74.122 |
| 3 | 108.612 | 78.770 | blq | 63.146 |
| 4 | 90.707 | 66.608 | blq | 52.438 |
| 6 | 93.453 | 60.975 | blq | 51.476 |
| 8 | 89.203 | 45.012 | blq | 44.738 |
| 12 | 80.474 | 44.169 | blq | 41.548 |
| 24 | 53.010 | 27.450 | blq | 26.820 |

TABLE 2
Table 2: Test data of a commercially available standard
RLD

| Time (h) | 7MW1103 | 7FW1121 | Mean |
|---|---|---|---|
| 0 | blq | blq | n.c. |
| 0.25 | 2.224 | 10.726 | 6.475 |
| 0.5 | 2.355 | 51.972 | 27.164 |
| 0.75 | blq | 70.389 | 35.195 |
| 1 | b1q | 75.391 | 37.696 |
| 1.5 | 2.216 | 335.451 | 168.834 |
| 2 | 2.667 | 234.060 | 118.364 |
| 3 | 2.747 | 313.750 | 158.249 |
| 4 | blq | 387.835 | 193.918 |
| 6 | 2.604 | 309.282 | 155.943 |
| 8 | blq | 34.821 | 17.411 |
| 12 | 2.534 | 48.842 | 25.688 |
| 24 | blq | 20.124 | 10.062 |

FIG. 7 is a schematic diagram of plasma drug concentration curves generated by combining the data in Table 1 and Table 2.

Wherein, it can be known from the figure of the commercially available control drug (RLD)—solid dots and the experimental tablet body of the invention (T2)—hollow squares that during a process of the drug of the invention being released in the blood, from 0.75 hours to 24 hours, the drug of the invention is released continuously and stably. Compared with the commercially available control drug (RLD)—solid dots, its concentration in the blood changes greatly within the same time. In this way, the drug structure of the invention can prevent symptoms from being difficult to control due to large fluctuations in a concentration of the drug in the blood. Compared with conventional drugs, the symptoms can be controlled more effectively without causing side effects.

Summing up the above, by forming the tablet body 1 through mixing the first excipient with the first penetration accelerator, and coating the outer layer of the tablet body with the coating layer 2 formed by mixing the second penetration accelerator with the peptide, thereby enabling the tablet body 1 containing the peptide to quickly settle to a bottom of a stomach, and at the same time, to quickly attach to the bottom of the stomach flatly, the peptide is attached to a stomach wall, and the excipient is covered on the peptide. Finally, a pH value of local areas in the stomach can be changed, thereby enabling the peptide to be stably absorbed through the stomach.

The above-mentioned embodiments are merely exemplification to illustrate the principles and efficacies of the invention, and are not used to limit the invention. Any person having ordinary skill in the art can modify the above-mentioned embodiments without departing from the spirit and scope of the invention. Therefore, the scope of protection of the rights of the invention should be as listed in the appended claims hereinafter.

What is claimed is:

1. An oral peptide drug structure comprising:
a tablet body made of a first excipient mixing with a first penetration accelerator; and
a coating layer made of a second penetration accelerator mixing with a peptide, and the coating layer being coated on an outer layer of the tablet body.

2. The oral peptide drug structure as claimed in claim 1, wherein the tablet body is further mixed with a second excipient and a third excipient.

3. The oral peptide drug structure as claimed in claim 2, wherein a weight percentage of each of the excipients in the tablet body is: a weight percentage of the first excipient is 0.5-5% (colloidal silica, SiO2), a weight percentage of the second excipient is 15-23% (microcrystalline cellulose, MCC), and a weight percentage of the third excipient is 7-12% (croscamellose sodium).

4. The oral peptide drug structure as claimed in claim 3, wherein a weight percentage of the first penetration accelerator is 62-73%.

5. The oral peptide drug structure as claimed in claim 4, wherein the first excipient, the second excipient, the third excipient and the first penetration accelerator are formed viscously and shaped by mixing with a first adhesive, wherein a weight percentage of the first adhesive in the tablet body is 0.13-0.5%.

6. The oral peptide drug structure as claimed in claim 5, wherein the peptide in the coating layer is mixed with a fourth excipient, and then mixed with the second penetration accelerator, and then mixed with a fifth excipient already mixed with a sixth excipient.

7. The oral peptide drug structure as claimed in claim 6, wherein a weight percentage of each of the excipients in the coating layer is: a weight percentage of the fourth excipient is 20-25% (microcrystalline cellulose, MCC), a weight percentage of the fifth excipient is 0.5-5% (colloidal silica, SiO2), a weight percentage of the sixth excipient is 5-9.5% (croscamellose sodium), and a weight percentage of the peptide in the coating layer is 1.2-13.3%.

8. The oral peptide drug structure as claimed in claim 7, wherein a weight percentage of the second penetration accelerator is 62-73%.

9. The oral peptide drug structure as claimed in claim 8, wherein the fourth excipient, the fifth excipient, and the sixth excipient are mixed with a second adhesive to form a viscous state and coated on the outer layer of the tablet body, wherein a weight percentage of the second adhesive in the coating layer is 0.13-0.2%.

10. A manufacturing method of the oral peptide drug structure as claimed in claim 9, wherein the method comprises:
a. mixing the first penetration accelerator with the first excipient to form a first mixture;
b. mixing the second excipient with the third excipient to form a second mixture;
c. mixing the first mixture with the second mixture to form a third mixture;
d. mixing the third mixture with the first adhesive to form a viscous tablet body, and drying it to take shape;
e. mixing the peptide with the second penetration accelerator to form a fourth mixture;
f. mixing the fifth excipient with the sixth excipient to form a fifth mixture;
g. mixing the fourth mixture with the fifth mixture in a dispersion manner to form a sixth mixture; and
h. mixing the sixth mixture with the second adhesive to form a seventh mixture to form a viscous state and coating the tablet body for performing drying operation.
